(19) United States
(12) Patent Application Publication
(10) Pub. No.: US 2003/0009184 A1
(43) Pub. Date: Jan. 9, 2003

(54) Catheter having variable wire size radiopaque braid

(75) Inventor: Henry J. Pepin, Loretto, MN (US)

Correspondence Address: Robert E. Atkinson, CROMPTON, SEAGER & TUFTE, LLC, Suite 895, 331 Second Avenue South, Minneapolis, MN, 55401-2246, US

(73) Assignee: SciMed Life Systems, Inc.

(21) Appl. No.: 09/898,687

(22) Filed: Jul. 3, 2001

(51) Int. Cl. A61B 17/22

(52) U.S. Cl. 606/159; 606/194; 604/103.09; 604/103.1

(57) ABSTRACT

A thin-walled reinforced catheter shaft having polymers that are reinforced with a variable wire size radiopaque braid which maintains the thin-wall of the shaft while providing improved properties in terms of radiopacity, kink resistance, tortional rigidity, column strength and burst strength.

### FIELD OF THE INVENTION

The present invention generally relates to intravascular medical devices. More specifically, the present invention relates to intravascular catheters such as guide and diagnostic catheters.

### BACKGROUND OF THE INVENTION

Intravascular catheter shafts commonly incorporate a reinforcement layer such as a stainless steel wire braid to enhance the strength of the shaft. Generally speaking, however, stainless steel wire braid is not highly radiopaque, and therefore is not highly visible using conventional x-ray radiographic visualization techniques.

### SUMMARY OF THE INVENTION

The present invention addresses this problem by providing, for example, an intravascular catheter having a reinforced elongate shaft which combines high strength (e.g., stainless steel) wires and highly radiopaque (e.g., tungsten) wires in an interwoven braid. The high strength wires provide torque, column strength and burst strength to the shaft, while the highly radiopaque wires provide enhanced radiopacity. The radiopaque wires have a diameter which is preferably less than the diameter of the high strength wires to avoid compromising the thin walls of the shaft.

### BRIEF DESCRIPTION OF THE DRAWINGS

FIG. 1 is a plan view of an intravascular catheter in accordance with an embodiment of the present invention;

FIG. 2 is a cross-sectional view taken along line 2-2 in FIG. 1;

FIG. 3 is a schematic illustration of the braid reinforcement pattern used in the intravascular catheter shown in FIG. 1; and

FIG. 4 is a cross-sectional view taken along line 4-4 in FIG. 1.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

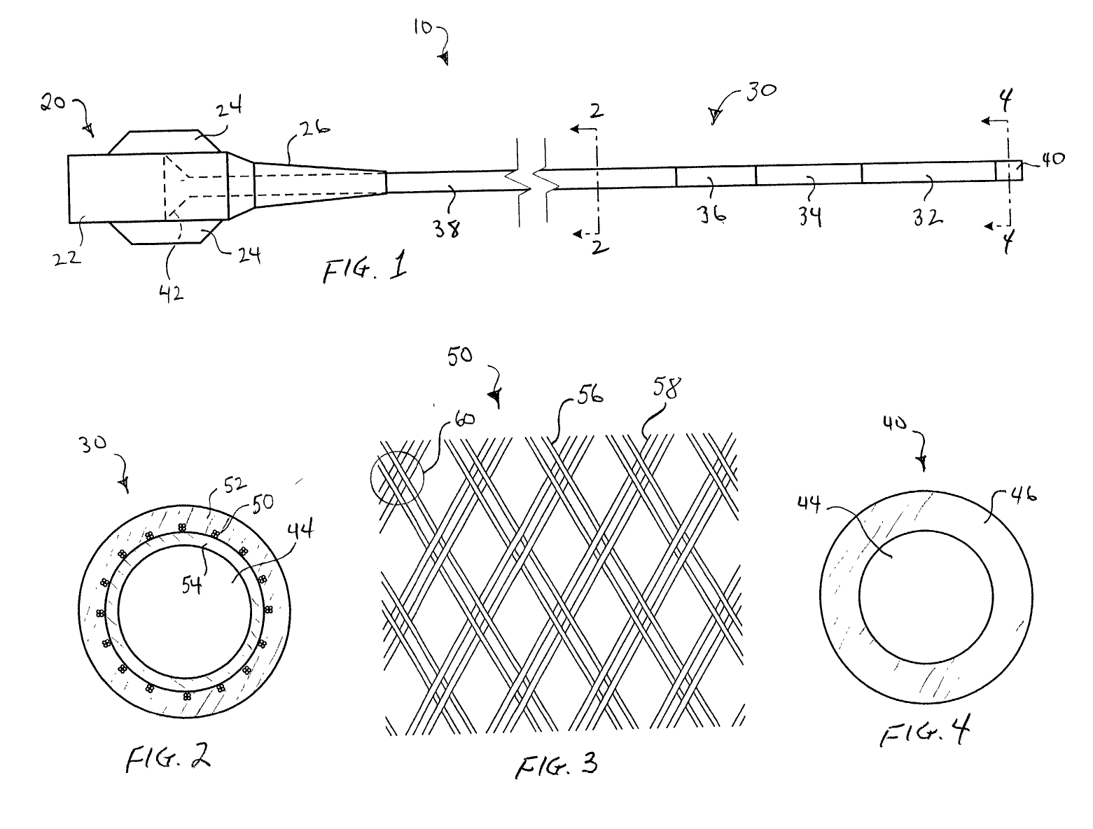

Refer now to FIG. 1 which illustrates a plan view of an intravascular catheter 10 in accordance with an embodiment of the present invention. Intravascular catheter 10 may comprise a wide variety of intravascular catheters such as a coronary guide or diagnostic catheter as shown. However, those skilled in the art will recognize that the principles and concepts described herein may be applied to virtually any intravascular catheter including balloon catheters, atherectomy catheters, etc. Except as described herein, the catheter 10 may be manufactured using conventional techniques and may be used in accordance with the intended clinical application.

In this particular example, the intravascular catheter 10 includes an elongate shaft 30 having a proximal end and a distal end. A hub and strain relief assembly 20 is connected to the proximal end of the elongate shaft 30. A proximal flared portion 42 of the elongate shaft 30 mechanically enhances the bond to the hub and strain relief assembly 20. The hub and strain relief assembly 20 includes a main body portion 22, a pair of flanges 24 to facilitate gripping and manipulation of the catheter 10, and a strain relief 26 to reduce the likelihood of kinking between the relatively stiff body portion 22 and the relatively flexible shaft 30. The hub and strain relief assembly 20 may be of conventional design and may be connected to the proximal end of the elongate shaft 30 utilizing conventional techniques.

The elongate shaft 30 includes a series of shaft segments which generally increase in flexibility toward the distal end of the elongate shaft 30. In this particular embodiment, the elongate shaft 30 includes a first shaft segment 32, a second shaft 34, a third shaft segment 36, and a forth shaft segment 38. The elongate shaft 30 also includes a distal atraumatic tip 40 and a proximal flared portion 42. The various shaft segments 32/34/36/38 are described in more detail with reference to FIG. 2, and the distal tip portion is described in more detail with reference to FIGS. 2 and 4.

Refer now to FIG. 2 which illustrates a cross-sectional view of the elongate shaft 30 taken along line 2-2 in FIG. 1. The cross-sectional view of the elongate shaft 30 shown in FIG. 2 is representative of the construction of each of the shaft segments 32/34/36/38 in addition to the proximal portion of distal tip 40. The distal portion of the distal tip 40 is represented by the cross-sectional view illustrated in FIG. 4 taken along line 4-4 in FIG. 1.

With continued reference to FIG. 2, the elongate shaft 30 includes an outer layer 52, an inner layer 54, and a reinforcement layer 50 disposed therebetween. The inner layer 54 defines a lumen 44 which extends through the entire length of the elongate shaft 30 and is in fluid communication with a lumen (not shown) extending through the hub assembly 20.

The inner layer 54 may comprise a lubricous polymeric material such as PTFE having an inside diameter of approximately 0.070 inches and a wall thickness of approximately 0.001 inches. The outer layer 52 may comprise a thermoplastic polymer such as a co-polyester thermoplastic elastomer (TPE) available under the tradename Arnitel. The outer layer 52 may have an inside diameter roughly corresponding to the outside diameter of the inner layer 54 and a wall thickness of approximately 0.005 inches. The reinforcement layer 50 is described in more detail with reference to FIG. 3.

The hub and strain relief 20 may have a length of approximately 2.10 inches and the elongate shaft 30 may have an overall length of approximately 39.1 inches. The distal tip segment 40 may have a length of approximately 0.130 inches, with the proximal 0.080 inches having a cross-section as shown in FIG. 2, and the distal 0.050 inches having a cross-section as shown in FIG. 4. The first shaft segment 32 may have a length of approximately 0.60 inches, the second shaft segment 34 may have a length of approximately 0.40 inches, the third shaft segment may have a length of approximately 0.030 inches, and the fourth shaft segment 38 may have a length of approximately 16.0 inches.

As mentioned previously, the various shaft segments 32/34/36/38 gradually decrease in stiffness toward the distal end of the elongate shaft 30. The decrease in stiffness may be provided by varying the hardness of the outer layer 52 corresponding to each shaft segment 32/34/36/38. For example, the distal unreinforced portion of the tip 40 may comprise a soft thermoplastic elastomer (TPE) sold under the name Hytrel having a hardness of 30D. To facilitate radiographic visualization, the unreinforced portion of the distal tip 40 may be loaded with 50% bismuth subcarbinate.

The outer layer 52 of the first shaft segment 32 and the proximal reinforced portion of the distal tip 40 may be formed of a TPE polymer sold under the tradename Arnitel having a hardness of 46D. The outer layer 52 of the second shaft segment 34 may be formed of a TPE polymer available under the tradename Arnitel having a hardness of 55D. The outer layer 52 of the third shaft segment 36 may be formed of a TPE polymer available under the tradename Arnitel having a hardness of 68D. The outer layer 52 of the fourth shaft segment 38 may be formed of a TPE polymer available under the tradename Arnitel having a hardness of 74D mixed with 6% liquid crystal polymer (LCP).

With reference to FIG. 3, the reinforcement layer 50 comprises an interwoven metal braid comprising a first wire or pair of wires 56 wound in a first helical direction and a second wire or pair of wires 58 wound in a second helical direction different from the first helical direction. The first wire or pair of wires 56 may comprise a highly radiopaque metal such as a tungsten having a relatively small diameter, and the second wire or pair of wires 58 may be formed of a high strength metal such as stainless steel having a relatively large diameter. The highly radiopaque wire or wires 56 provide clear visualization of substantially the entire length of the elongate shaft 30 during x-ray visualization. The high strength wire or wires 58 provide tortional rigidity, column strength and burst strength to the elongate shaft 30.

The highly radiopaque wire or wires 56 preferably have a diameter which is less than the diameter of the high strength wire or wires 58 such that the radiopaque wire or wires 56 do not significantly contribute to the overall wall thickness of the elongate shaft 30. Also preferably, the radiopaque wire or wires 56 and the high strength wire or wires 58 are wound in a two-over-two pattern as shown in FIG. 3 with an intersection 60 count or pic count of about 60 intersections per inch. The braid reinforcement 50 may comprise, for example, 16 strands of tungsten wire having a diameter approximately 0.0015 inches interwoven in a two-over-two pattern with 16 strands of stainless steel wire 58 having a diameter of approximately 0.0020 inches.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. An intravascular catheter, comprising:
an elongate shaft having a proximal end, a distal end and a lumen extending therethrough, the shaft including an inner layer, an outer layer, and a reinforcement layer disposed therebetween, the reinforcement layer comprising a first wire wound in a first direction and a second wire wound in a second direction different from the first direction, the first wire interwoven with the second wire, the first wire having a first diameter and the second wire having a second diameter less than the first diameter.

2. An intravascular catheter as in claim 1, wherein the first wire comprises a stainless steel metal wire to provide strength to the shaft.

3. An intravascular catheter as in claim 2, wherein the second wire comprises a highly radiopaque metal wire to provide radiopacity to the shaft.

4. An intravascular catheter as in claim 3, wherein the reinforcement layer further comprises a third wire running parallel and adjacent to the first wire.

5. An intravascular catheter as in claim 4, wherein the third wire has a diameter equal to the diameter of the first wire.

6. An intravascular catheter as in claim 5, wherein the reinforcement layer further comprises a fourth wire running parallel and adjacent to the second wire.

7. An intravascular catheter as in claim 6, wherein the fourth wire has a diameter equal to the diameter of the second wire.

8. An intravascular catheter, comprising:
an elongate shaft having a proximal end, a distal end and a lumen extending therethrough, the shaft including an inner layer, an outer layer, and a reinforcement layer disposed therebetween, the reinforcement layer comprising a first wire wound in a first direction and a second wire wound in a second direction different from the first direction, the first wire interwoven with the second wire, the first wire comprising stainless steel and the second wire comprising a highly radiopaque metal.

9. An intravascular catheter as in claim 8, wherein the first wire has a first diameter and the second wire has a second diameter less than the first diameter.

10. An intravascular catheter as in claim 8, wherein the reinforcement layer further comprises a third wire running parallel and adjacent to the first wire.

11. An intravascular catheter as in claim 10, wherein the third wire has a diameter equal to the diameter of the first wire.

12. An intravascular catheter as in claim 10, wherein the reinforcement layer further comprises a fourth wire running parallel and adjacent to the second wire.

13. An intravascular catheter as in claim 12, wherein the fourth wire has a diameter equal to the diameter of the second wire.

14. An intravascular catheter, comprising:
an elongate shaft having a proximal end, a distal end and a lumen extending therethrough, the shaft including an inner layer, an outer layer, and a reinforcement layer disposed therebetween, the reinforcement layer comprising a first pair of wires wound in a first direction and a second pair of wires wound in a second direction different from the first direction, the first pair of wires interwoven with the second pair of wires, the first pair of wires comprising stainless steel wires having a first diameter, and the second pair of wires comprising highly radiopaque metal wires having a second diameter less than the first diameter.

15. An intravascular catheter as in claim 14, wherein the elongate shaft is sized to navigate vascular pathways.

16. An intravascular catheter as in claim 15, wherein the outer layer includes multiple sections of distally decreasing stiffness.

17. An intravascular catheter as in claim 16, wherein the shaft includes a distal tip having a proximal portion and a distal portion, the distal portion of the tip comprising a flexible polymer without the inner layer or the reinforcement layer.

18. An intravascular catheter as in claim 17, wherein the flexible polymer of the distal portion of the distal tip is readily bondable to the outer layer.
